(19) United States
(12) Patent Application Publication
(10) Pub. No.: US 2010/0048523 A1
(43) Pub. Date: Feb. 25, 2010

(54) Compounds, Compositions and Methods for Treating Hormone-Dependent Maladies

(76) Inventors: Kurtis E. Bachman, Chester Spring, PA (US); Ben Ho Park, Baltimore, MD (US)

Correspondence Address: DLA PIPER LLP (US), 4365 EXECUTIVE DRIVE, SUITE 1100, SAN DIEGO, CA, 92121-2133, US

(21) Appl. No.: 12/296,277

(22) PCT Filed: Apr. 6, 2007
(86) PCT No.: PCT/US07/66167

§ 371 Date: Oct. 7, 2009

Related U.S. Application Data
(60) Provisional application No. 60/789,885, filed on Apr. 7, 2006.

(51) Int. Cl. A61K 31/58; A61P 35/00; A61P 15/00; A61P 15/18

(52) U.S. Cl. 514/174

(57) ABSTRACT

A composition and method of using are provided, which composition includes: at least one compound selected from the group including a compound having formula (I), a salt thereof, a prodrug thereof, a compound having formula (II), a salt thereof, a prodrug thereof, and a combination thereof, the compounds being described herein, and a combination thereof, and at least one pharmaceutically acceptable carrier; wherein the composition is suitable for administration to a mammal. Methods of using the compound are also provided.

### CROSS REFERENCE TO RELATED APPLICATIONS

This application is an International Patent Application based on and claims priority to U.S. Provisional Application Ser. No. 60/789,885, filed Apr. 7, 2006, the entire contents of which are hereby incorporated by reference.

### BACKGROUND

### DISCUSSION OF THE BACKGROUND

Hormonal therapy has made a tremendous impact on the management of hormone-dependent maladies such as, for example, breast cancers expressing estrogen receptors. Epidemiological data shows that prolonged estrogen exposure can increase the risk of breast cancer. ER alpha (ERα)/PR+breast cancers respond to hormonal therapies. The selective estrogen receptor modulator (SERM), Tamoxifen, is known to be effective in reducing the morbidity and mortality associated with estrogen receptor and/or progesterone receptor (ER/PR) positive breast cancers. ER/PR positive breast cancers account for roughly 50-70% of all breast cancers in the United States. Interestingly, Tamoxifen also prevents breast cancers in women at high risk, which emphasizes the carcinogenic potential of cumulative estrogen exposure. Despite its widespread use, and its proven ability to treat and prevent local (primary) and metastatic breast cancers, de novo and acquired resistance to Tamoxifen, and the increased risk of endometrial cancer in women undergoing Tamoxifen therapy, are still major clinical problems. Therefore, it is important to identify and target new pathways for the treatment of hormonally resistant breast cancers and hormone dependent maladies.

The standard of care for women with locally involved (curable) ER/PR positive breast cancers is to take Tamoxifen for five years as adjuvant therapy. Tamoxifen is also indicated in metastatic ER/PR positive breast cancers. Tamoxifen has been shown to reduce the risk of recurrence when used in the adjuvant setting by as much as 47%. However, Tamoxifen is not a pure anti-estrogen, but is a SERM as mentioned above. Clinically, this means that Tamoxifen has selective estrogenic effects, depending on the tissue type expressing the estrogen receptor. Tamoxifen exhibits anti-estrogenic effects on breast tissue, but estrogenic effects on endometrium, bone, and the cardiovascular system. Tamoxifen, like estrogen, can prevent osteoporosis, but it also stimulates endometrial tissue leading to a small increased risk of endometrial cancers. This has limited the ability of this drug to be taken long term, since the risk of developing endometrial cancer will eventually outweigh any beneficial effects in preventing breast cancer recurrence. It has also been demonstrated in the metastatic setting that prolonged exposure to Tamoxifen will almost always give rise to Tamoxifen resistant breast cancers. An equally intriguing observation is that some of these Tamoxifen resistant breast cancers can become Tamoxifen dependent, since withdrawal of Tamoxifen often leads to regression of the metastatic disease. Tamoxifen resistance is a poorly understood process and may in some cases involve the overexpression of oncogenes. Of the genes possibly involved with Tamoxifen resistance, p21 is a cyclin dependent kinase (CDK) inhibitor originally cloned as a downstream effector of p53. Previous studies have suggested that the loss of CDK inhibitors may confer Tamoxifen resistance. Loss of p21 expression is frequent in breast cancers.

Tamoxifen has the following structure:

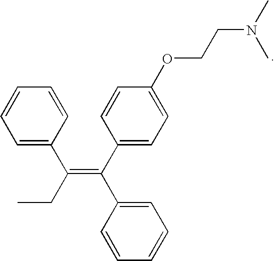

Although Tamoxifen remains the standard of care for hormone responsive breast cancer in the United States, new therapies have evolved, and some may eventually supplant Tamoxifen in specific clinical settings. For example, other SERMs such as Raloxifene appear to inhibit breast cancer growth but do not exhibit a stimulatory effect on uterine tissues. Drug resistance to this compound can still develop, however. Faslodex, (ICI 182,780) a pure anti-estrogen, has also demonstrated potential as a breast cancer therapeutic in ER/PR positive diseases, although its clinical use has remained limited. Recent data suggests that aromatase inhibitors (AIs) are superior to Tamoxifen in the metastatic and adjuvant settings, but these drugs are usually only indicated for post-menopausal women with hormone responsive disease since ovarian production of estrogen is a contraindication to AIs in pre-menopausal women. The long-term side effects of total estrogen depletion induced by AIs are not yet known.

The present inventors have found that it would be desirable to obtain a compound or compound that deliver the therapeutic benefits of estrogen, while still selectively blocking the harmful effects of estrogen on breast and endometrial tissues. It may be that a combination of SERMs along with new drugs that target SERM resistant breast cancers would allow one to target pathogenic elements of estrogen receptor signaling, while leaving the beneficial components intact.

### DESCRIPTION OF THE FIGURES

FIG. 1 shows stably transfected ERα IRES puro2 clones express high levels of ERα. MCF-10A cells, p21+/− and p21−/− cells were transfected with an ERU wild type cDNA and selected in puromycin and multiple clones were isolated. Cell lysates were made and subjected to Western blotting using a monoclonal human ERα antibody. A monoclonal anti-GAPDH antibody was used as a loading control. “ERIN” denotes “Estrogen Receptor In Normal” MCF-10A cells, while p21+/− and p21−/− are MCF-10A p21 heterozygous and homozygous clones respectively. ERα positive heterozygous clones were derived from separate p21+/− clones. ERα positive homozygous clones were derived from separate p21−/− clones (1.6 and 1.8), which were themselves derived from separate p21+/− heterozygous clones, to minimize the possibility of clonal artifacts.

FIG. 2 shows that p21 knock out cell lines grow in response to Tamoxifen. ERα positive MCF-10A (ERIN#9), pIRESpuro2 transfection control (−) control, and ERα positive MCF-10A p21−/− (1.6#4) cell lines were seeded in assay media (no EGF) and exposed to estrogen (E2), vehicle only, estrogen and Tamoxifen (E2+Tam), estrogen and Faslodex (E2+ICI), Tamoxifen alone (Tam) and Faslodex alone (ICI). Cells were grown for 1 week and stained with crystal violet. Results are representative of multiple clones including heterozygous controls, and assays were repeated three times for each clone.

FIG. 3A shows metastatic breast cancer (skin) in 2003. The nodule in the picture is a recurrence of a previous cancer, growing in the presence of Tamoxifen.

FIG. 3B shows the same area as in FIG. 4A one month after Tamoxifen cessation. The nodule went away after cessation of Tamoxifen treatment.

FIG. 4 shows a case report showing cancerous tissue in the breast, colon and skin of a female subject from 1991 to 2003. The H&E staining in the left-hand column shows the blue (or dark) stained cancer nuclei. The right-hand column shows a red (or darker) staining specific to the p21 protein.

FIG. 5 shows that loss of p21 causes Tamoxifen to act as an estrogen.

FIG. 6 shows a schematic of high-throughput drug screening using isogenic knock-out cancer cell lines.

FIG. 7 shows % survival of parental (Erin) and p21 knock-out (Erik, “Estrogen Receptor in Knockout”) cell lines vs. concentration of the S&MH4 Compound 233-E09 (compound having the formula (I′)) in accordance with one embodiment of the invention.

FIG. 8 shows % survival of parental (Erin) and p21 knock-out (Erik) cell lines vs. concentration of the Compound 233-D4 (compound having the formula (II)) in accordance with another embodiment of the invention.

FIG. 9 shows % survival of parental (Erin) and p21 knock-out (Erik) cell lines vs. concentration of a known, non-specific analog (Compound 234-B6, MW 359.51, structure not shown) as a comparative example.

FIG. 10 shows % survival of parental (Erin) and p21 knock-out (Erik) cell lines vs. concentration of a comparative compound, a known progesterone derivative used in oral contraceptives, in the control of menstruation, and in the treatment of abnormal uterine bleeding (MW 298.42 g/mol) having the following structure:

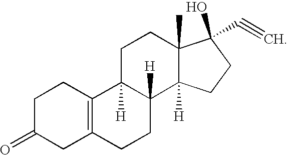

FIG. 11 shows % survival of parental (Erin) and p21 knock-out (Erik) cell lines vs. concentration of a comparative compounds, a known synthetic progestational hormone often used in mixtures with estrogens as an oral contraceptive (MW 284.45 g/mol) having the following structure:

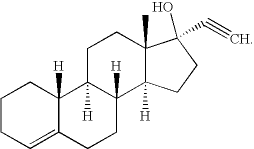

FIG. 12 shows % survival of parental (Erin) and p21 knock-out (Erik) cell lines vs. concentration of a comparative compound, a known estrogen derivative, which acts as an estrogen replacement to treat vaginal symptoms (MW 266.34 g/mol) having the following structure:

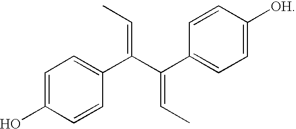

FIG. 13 shows % survival of parental (Erin) and p21 knock-out (Erik) cell lines vs. concentration of a comparative compound, a known anti-estrogen, Faslodex (fulvestrant) having the following structure:

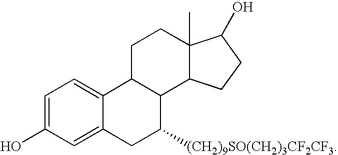

### BRIEF DESCRIPTION OF THE SEVERAL EMBODIMENTS

The present inventors have identified and isolated compounds that are capable of killing Tamoxifen resistant breast cancer cells. These drugs are particularly suited for the treatment of hormonally resistant diseases and for the treatment of hormone dependent maladies.

The compounds have the structures as shown below. One compound has the following formula (I):

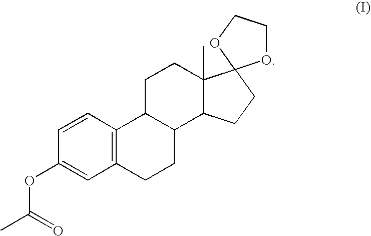

Another compound has the following formula (II):

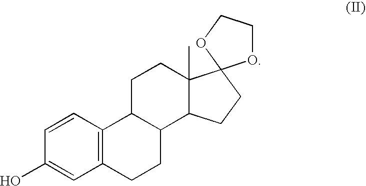

These compounds are suitable for the treatment of hormone dependent maladies and/or estrogen-related conditions including but not limited to hormone receptor positive cancers, vaginitis, osteoporosis, cardiovascular disease, and decreased fertility. These compounds selectively inhibit the growth of ER positive epithelial cells that have acquired resistance to current therapies. These compounds may also be used as contraceptives.

In one embodiment, steroidal (fused carbon-ring) compounds with molecular weights of 356.46 g/mol (compound having formula (I′), sometimes referred to herein as compound 233-E09, S&MH4, C₂₂H₂₈O₄) and 314.42 g/mol (compound having formula (H), sometimes referred to herein as compound 233-D4, C₂₀H₂₆O₃) target tamoxifen resistant ER positive cells. These compounds possess a unique spiro heteropentane ring with 2 oxygen atoms and the common carbon atom shared with the pentane ring of the steroidal backbone.

The compound having formula (I′) is shown below:

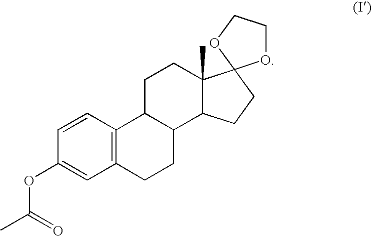

One embodiment relates to the use of cell lines that differ only with respect to resistance to act as phenotypic controls to discover compounds that selectively target a specific resistance pathway. It has been previously demonstrated that somatic cell deletion of the cyclin dependent kinase inhibitor p21 enables non-tumorigenic breast epithelial cells to use Tamoxifen as a growth stimulatory molecule. Ablation of p21 in the MCF-7 cell lines leads to a Tamoxifen resistance phenotype. Like the MCF-10A p21 −/− cells, deletion of p21 also confers an estrogenic growth response to Tamoxifen. Multiple independently derived clones are isolated and compared to their heterozygous and parental counterparts. The use of these isogenic somatic cell knock out cell lines allows one to find compounds that target the p21 pathway and therefore Tamoxifen resistance. These paired cell lines therefore serve as a “genetic” control for the drug screen outlined herein.

Phenotypic selection of Tamoxifen resistant MCF-7 cell lines has been previously described. Tamoxifen resistant clones are generated by culturing and single cell diluting MCF-7 in estrogen free Tamoxifen containing medium. Because there are likely many different genetic mechanisms leading to Tamoxifen resistance, ten to twenty clones are isolated for study. To ensure that genetic effectors other than p21 loss are occurring in these resistant clones, Western Blot analysis is performed to determine if p21 expression is lost in any of the resistant cell lines. Four Tamoxifen resistant clones have been generated, and the presence of p21 expression in these cell lines has been verified. These clones then form the basis for the initial drug screen targeting Tamoxifen resistance as described herein.

In the present application, high throughput drug screening is used with genetically and phenotypically defined Tamoxifen resistant breast cancer cell lines to identify new therapies for the treatment of hormonally resistant breast cancers and, in turn, other hormone dependent maladies. It has been previously shown that by using isogenic somatic cell knock out lines that differ only by the deletion of a specific gene, one can uncover compounds that target cells in a genotype-specific fashion. The genotypic changes behind Tamoxifen resistance, however, are largely unknown and likely involve multiple different mechanisms. The present inventors have found that deletion of p21 in MCF-10A cells confers a Tamoxifen resistance phenotype. The present inventors have also found that, in the breast cancer cell line MCF-7, Tamoxifen resistance can occur without p21 loss. The high-throughput cell-based chemical screen, as used herein, identifies compounds that selectively target the growth of Tamoxifen resistant cells. By screening cell lines that have acquired Tamoxifen resistance through the deletion of p21, and also cells that have spontaneously acquired this resistance through unknown mechanisms, compounds have been identified that selectively target the p21 -induced resistance pathway and the pathways that lead to spontaneous Tamoxifen resistance. These compounds that target both types of resistant cells are significant, inter alia, because they: (1) kill through a p21/Tamoxifen dependent mechanism versus a p21 “only” mechanism; and (2) are clinically relevant in targeting Tamoxifen resistance since loss of p21 expression is a common occurrence in human breast cancers.

Examples of hormonal-dependent maladies include cancer, tamoxifen-resistant cancer, tamoxifen resistant breast cancer, hormone-dependent breast cancer, hormone-dependent endometrial cancer, hormone-dependent ovarian cancer, estrogen receptor expressing cancer, progesterone receptor expressing cancer, vaginitis, vaginal dryness, post-menopausal vaginitis, osteoporosis, cardiovascular disease, decreased fertility, and infertility. Combinations are possible.

The compound may also be useful as a contraceptive.

The hormone-dependent cancer may be primary hormone-dependent cancer, recurrent hormone-dependent cancer, metastatic hormone-dependent cancer, and combinations thereof. The recurrence may be primary recurrence or metastatic recurrence.

The breast cancer may be primary, recurrent, and/or metastatic hormone-dependent breast cancer. Examples of breast cancers include ductal carcinoma and lobular carcinoma.

The endometrial cancer may be primary, recurrent, and/or metastatic hormone-dependent endometrial cancer. One example of endometrial cancer is adenocarcinoma.

The ovarian cancer may be primary, recurrent, and/or metastatic hormone-dependent ovarian cancer. Examples of ovarian cancers include mucinous carcinoma and serous carcinoma.

One of ordinary skill can readily diagnose hormonal-dependent maladies and would be able to determine when a subject is in need of treatment. For example, a cancer growing under the administration of Tamoxifen would be an example of a Tamoxifen resistant cancer.

The compound may be administered as a systemic or as a local treatment.

The compound may be administered in a manner similar to known hormone therapies. Hormone therapy desirably prevents estrogen or progesterone from stimulating cancer cells. It may suitably be used for those cancers that are hormone-receptor positive (for example, either estrogen-receptor positive or progesterone-receptor positive cancers), regardless of the size of the tumor and whether or not it has spread.

The compound may be administered as primary therapy for subjects for whom surgery or radiation therapy is not appropriate. Alternatively, the compound may be administered in combination with surgery, radiation or both (adjuvant therapy). Adjuvant therapy may be particularly beneficial for subjects who have microscopic evidence of the spread of cancer at the time of diagnosis. Such therapy may kill residual cancer cells before they have a chance to become clinically evident. Alternatively, the compound may be administered prior to local treatment or neoadjuvant therapy. The goal in such cases is usually to shrink locally advanced tumors (Stage III) to a size small enough for surgical or radiological therapy. Alternatively, in some cases of metastatic cancer, the compound may be administered not to cure but to improve quality of life and possibly prolong survival.

As used herein, the terms “treat”, “treating” and/or “treatment” refers to the action of the compound, prodrug, salt, or composition to improve or alter an outcome in a subject having a hormone dependent malady. The skilled artisan is aware that the improvement or alteration may be in whole or in part and may not be a complete cure. Treating may also comprise treating a subject at risk for developing a disease and/or condition.

The subject includes anyone at risk for or diagnosed with a hormonal-dependent malady. In one embodiment, the subject is a mammal, for example, a human or mouse.

The term “therapeutically effective amount” refers to an amount of the compound, prodrug, salt, or combination thereof which is effective, upon single or multiple dose administration or continuous administration, infusion or application to the patient, for the treatment of a hormone dependent malady. A therapeutically effective amount can be readily determined by the attending diagnostician, as one skilled in the art, by the use of known techniques and by observing results obtained under analogous circumstances. In determining the therapeutically effective amount or dose, a number of factors are considered by the attending diagnostician, including, but not limited to the subjects size, age, sex, and general health; the hormone dependent malady involved; the degree of or involvement or the severity of the disease; the response of the individual subject; the particular compound administered; the bioavailability characteristics of the preparation administered; the dose regimen selected; the use of concomitant medication; and other relevant circumstances.

A therapeutically effective amount of the compound may range from about 0.0001 milligram per kilogram of body weight per day (mg/kg/day) to about 10,000 mg/kg/day. Preferred amounts may range from about 0.001 to about 100 mg/kg/day. These ranges include all values and subranges therebetween, including 0.0001, 0.0005, 0.001, 0.005, 0.01, 0.05, 0.1, 0.5, 1, 2, 3, 4, 5, 6, 7, 8, 9, 10, 100, 1,000, 5,000, and 10,000 mg/kg/day, and any combination thereof.

The compound may be administered to the subject in any form or mode which makes the compound bioavailable in effective amounts, including orally, subcutaneously, intramuscularly, intravenously, transdermally, intranasally, rectally, topically, intramucosaly, intravaginally, parenterally, and the like. Given the teachings herein, one skilled in the art can readily select the proper form and mode of administration depending upon the compound selected, the malady to be treated, the stage of the disease, and other relevant circumstances.

The compound, prodrug, or salt can be administered alone or in the form of a pharmaceutical composition in combination with one or more pharmaceutically acceptable carriers so long as the composition is suitable for administration to a mammalian subject, and particularly a human. In one embodiment, the compound includes tautomeric forms, and prodrugs and salts of these forms are contemplated. Mixtures of one or more are possible.

The compound or composition may be suitably administered batchwise or by constant or periodic infusion over an extended period of time, for example, exceeding 24 hours, until the desired therapeutic, preventive, and/or inhibiting benefits are obtained.

The carrier is physiologically tolerable by a human and does not interfere with the intended effect of the active ingredient. Examples of the pharmaceutically acceptable carrier include water, physiological saline, ethanol, aqueous ethanol, dimethyl sulfoxide, castor oil, benzyl alcohol, benzyl benzoate, albumin, polyethylene glycol, cellulose, fatty acid, methylcellulose, dextrose, glycerol, mannitol, lactose, starch, magnesium stearate, sodium saccharin, or magnesium carbonate, or a combination thereof.

The suitability of particular carriers for inclusion in a given therapeutic composition may depend on the route of administration desired. For example, the composition may be prepared as liquid solution, suspension, emulsion, cream, inhalant, patch, implant, solid, tablet, pill, capsule, sustained release, or powder form. The composition may include such one or more additives or excipients such as binder, filler, preservative, stabilizing agent, emulsifier, wetting agent, emulsifying agent, stabilizing agent, pH buffering agent, and the like. Combinations are possible.

The composition may typically contain 1% to 95% by weight of active ingredient, be it the compound, salt, prodrug, or a combination thereof. This range includes all values and subranges therebetween, including 1, 2, 3, 4, 5, 6, 7, 8, 9, 10, 20, 30, 40, 50, 60, 70, 80, 90, and 95% by weight, and any combination thereof.

The IC₅₀ may range from 1 nanomolar to 1 micromolar (moles of active/liters of culture medium). This range includes 1, 2, 3, 4, 5, 6, 7, 8, 9, 10, 20, 30, 40, 50, 60, 70, 80, 90, 100, 200, 300, 400, 500, 600, 700, 800, 900, 1000 nM, and any combination thereof.

### EXAMPLES

Generation of Tamoxifen resistant human breast cancer cell lines: Tamoxifen resistance frequently develops when treating hormonally responsive breast cancers. Loss of the cyclin dependent kinase inhibitor, p21, is one genetic mechanism leading to Tamoxifen resistance and Tamoxifen growth stimulation in breast epithelial cells, and p21 loss is a frequent event in breast cancers. Drug resistant breast cancer cell lines can be generated through clonal selection in Tamoxifen containing media. Tamoxifen resistant breast cancer cell lines are generated and characterized using genetic and phenotypic selection with the ER alpha (ERα) positive breast cancer cell line, MCF-7. MCF-7 breast cancer cell lines are transfected using Fugene6 with p21 knock out vectors as previously described. Although this cell line is grossly aneuploid, karyotypic and SKY analyses have demonstrated that there are only two copies of chromosome 6 where the p21 gene resides (http://www.path.cam.ac.uk/˜pawefish/). Thus, stable p21 knock out clones are generated in the breast cancer cell line MCF-7. These p21−/− cell lines are then tested for their ability to resist Tamoxifen induced growth arrest upon exposure to Tamoxifen and estrogen. MCF-7 cells are grown in estrogen free, Tamoxifen containing media. Cultures are single cell diluted and Tamoxifen resistant clones are isolated and expanded. The expression of p21 is assessed in these resistant clones to ensure that other genetic mediators conferring a Tamoxifen resistant phenotype are generated.

The creation of a non-tumorigenic human breast epithelial cell line (MCF-10A) in which the p21 gene was deleted through targeted homologous recombination has been previously described. Because p21 has been implicated in Tamoxifen resistance, and the response of Tamoxifen in both parental MCF-10A cells and their p21−/− derivatives was assessed. MCF-10A cells are ERα negative and unresponsive to estrogen stimulation. In order to develop a model system to analyze the effects of estrogen and anti-estrogen signaling, MCF-10A cells and their p21−/− derivatives were stably transfected with a cDNA encoding for wild type ERα.

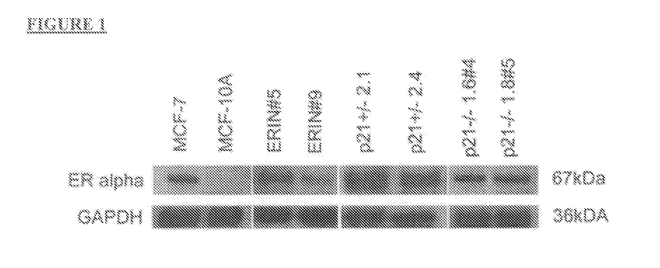

Unlike previous attempts to stably integrate ERα into the MCF-10A cell line, a cloning vector containing an internal ribosome entry site (IRES) was used to ensure high levels of wild type ERα expression. The full coding sequence of wild type ERα was PCR cloned into pIRESpuro2 (Clontech) and the absence of any coding mutations was verified by direct sequencing. This construct was then stably transfected into parental MCF-10A, p21+/− and p21−/− clones to obtain cell lines that expressed levels of ERα that were comparable to the MCF-7 ERα positive breast cancer cell line as shown in FIG. 1. The previously published MCF-10A cell line was obtained, which was transfected with a traditional non-IRES ERα vector, and it was found to produce almost undetectable levels of ERα by Western Blot analysis.

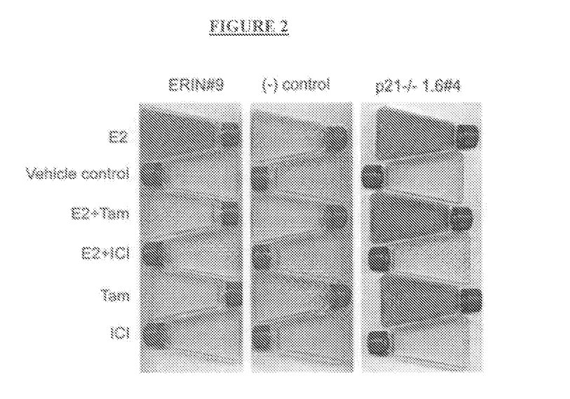

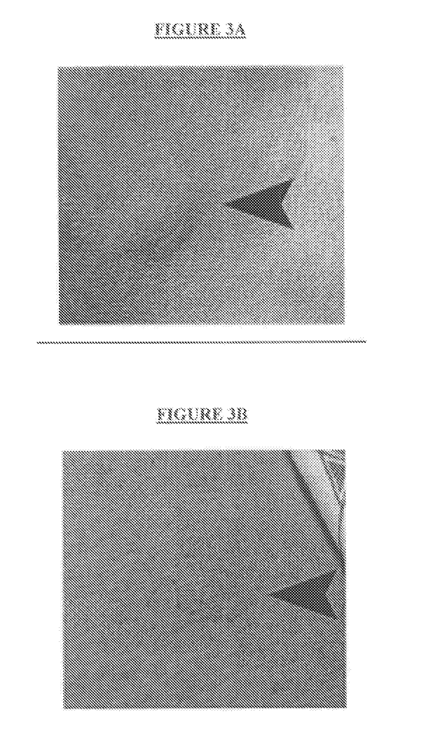

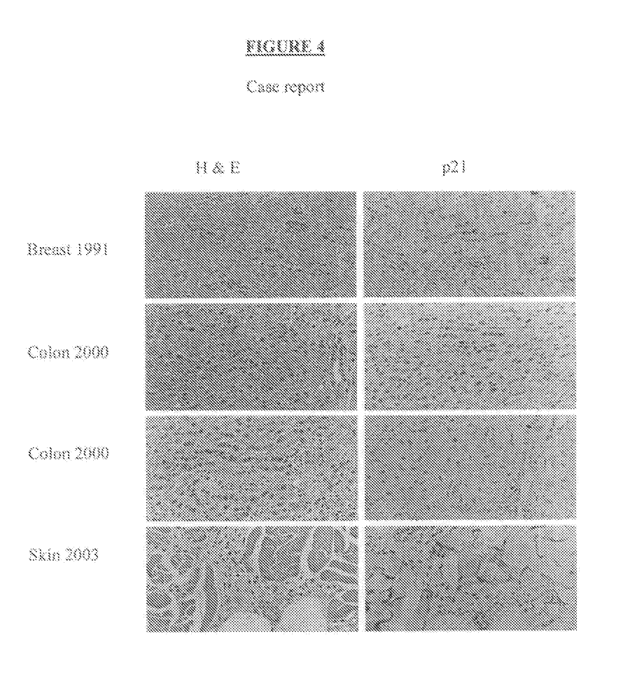

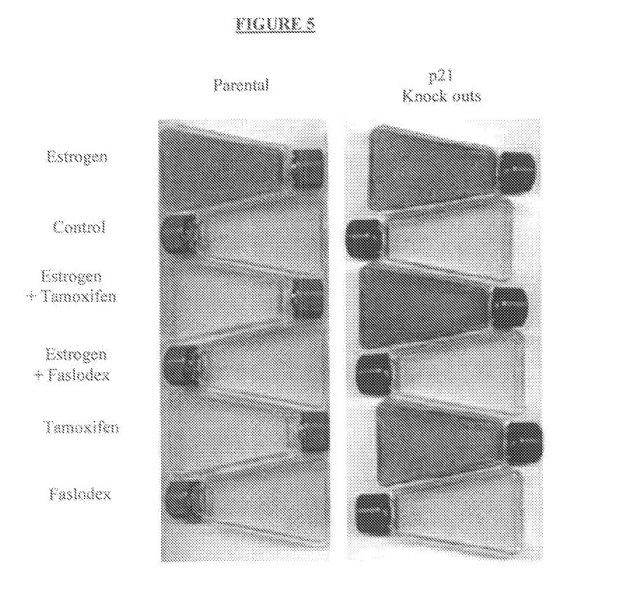

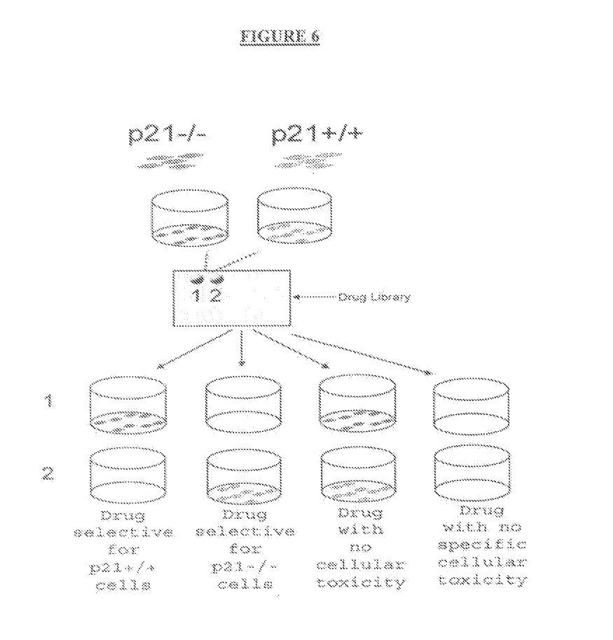

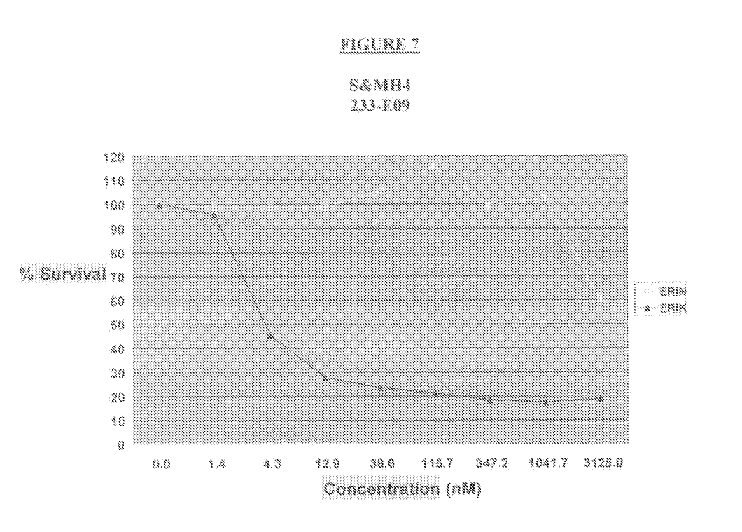

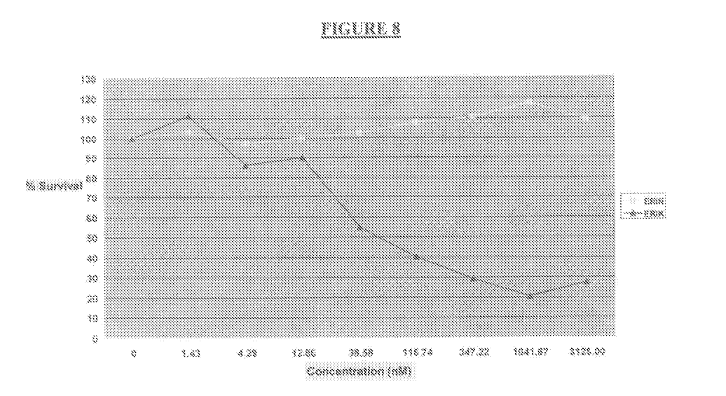

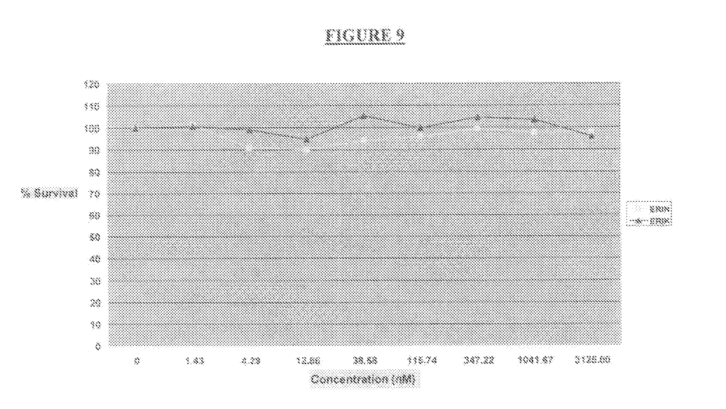

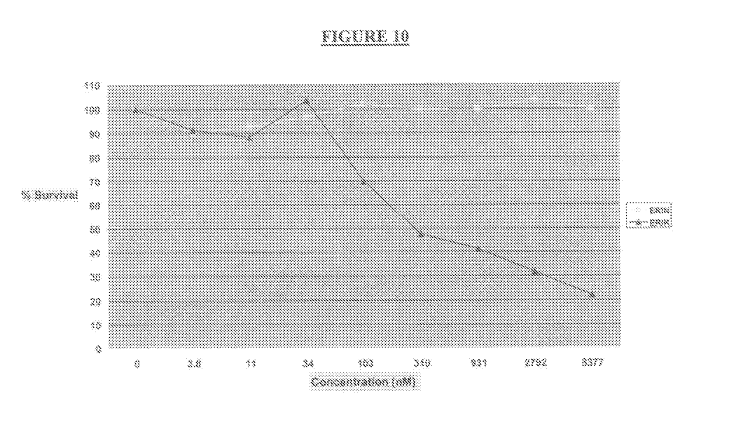

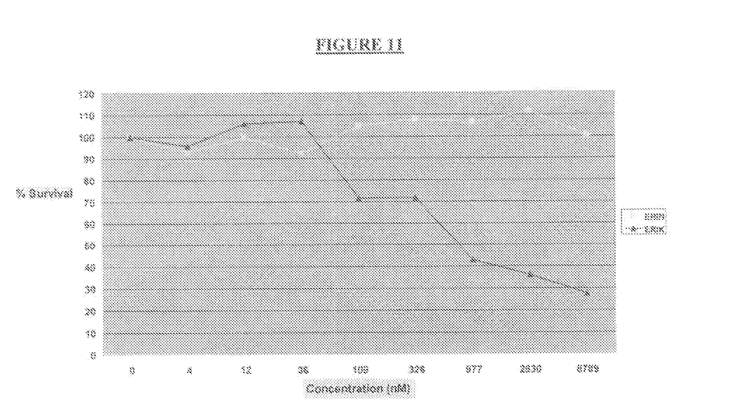

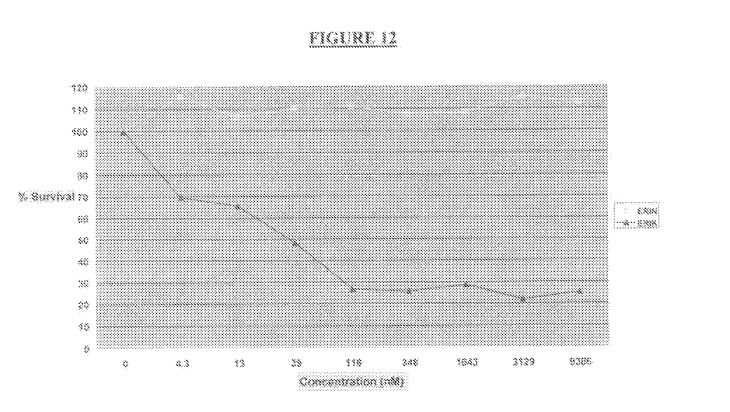

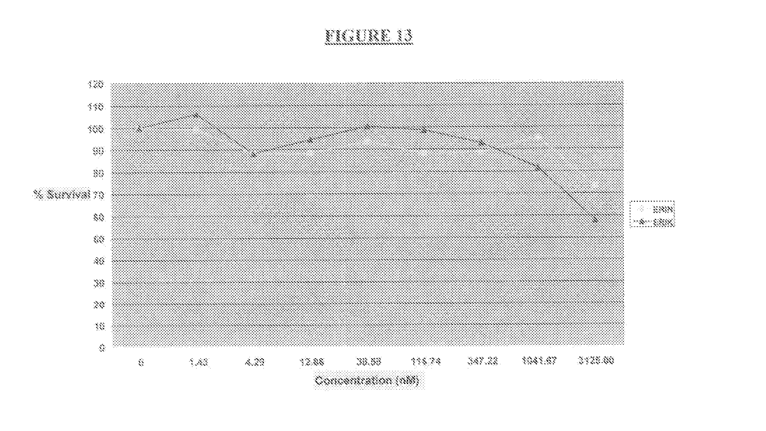

Cell lines were generated and grown with charcoal stripped dextran treated serum (Hyclone) and phenol red free medium to avoid any possible growth effects of estrogen in the medium. As shown in FIG. 2, the MCF-10A cells stably overexpressing ERα proliferated in response to estrogen, and this effect was inhibited by Tamoxifen and the pure anti-estrogen Faslodex (ICI 182,780). MCF-10A p21+/− heterozygous clones displayed an identical pattern of growth to these reagents. Surprisingly, MCF-10A p21 knock out cell lines were resistant to the growth inhibitory effects of Tamoxifen, and moreover, these cell lines grew in response to this SERM (FIG. 2).

The SERM Raloxifene behaved identically to Tamoxifen in the p21+/+, p21+/−, and p21−/− ERα positive cell lines, though its effect on cell proliferation in p21 null cells was not as pronounced as Tamoxifen's. In this regard, the p21 null cells do not display the same phenotype as endometrial cells, as endometrial cells are growth-inhibited by Raloxifene. Based on its known mechanism of decreasing ERα levels, the pure anti-estrogen Faslodex could still inhibit the proliferative effects of estrogen in p21−/− cell lines.

After two rounds of gene targeting, a number of independently derived p21 null MCF-7 cell lines similar to the p21−/− MCF-10A cells are generated. These cell lines are then tested for their phenotypic response to estrogen, Tamoxifen, and estrogen with Tamoxifen to determine if MCF-7 p21 null cells exhibit Tamoxifen resistance. Cells are also cultured with Faslodex, similar to the growth conditions described herein for MCF-10A cells.

Drug library screening to isolate compounds that can selectively inhibit the growth of Tamoxifen resistant breast cancer: Chemical drug libraries were purchased and used in a high throughput screen to select compounds that specifically kill or inhibit the growth of Tamoxifen resistant MCF-7 breast cancer cell lines, but not their MCF-7 Tamoxifen sensitive parental counterparts. The phenotypic generation of Tamoxifen resistant breast cancer cell lines in vitro may or may not reflect the genetic events that occur in vivo. However, loss of p21 expression has been demonstrated in human breast cancers, and the present compounds are shown herein to specifically kill p21−/− MCF-10A breast cancer cell lines, but not their p21 +/+ counterparts. This secondary screen increases the likelihood that the present compounds and compositions target clinically relevant Tamoxifen resistant breast cancers.

Tamoxifen resistant clones established herein and isogenic parental clones are used as target and control lines, respectively. Cells are maintained and screened in DMEM F12 medium (Invitrogen/Gibco, Rockville, Md.) supplemented with 5% (vol/vol) estrogen-free serum, and 100 units/ml penicillin and 0.1 mg/ml streptomycin. Medium for Tamoxifen resistant clones are supplemented with 100 nM Tamoxifen (Sigma, St. Louis, Mo.).

Screening steps are performed using a Biomek FX Laboratory Automation Workstation (Beckman Coulter, Inc., Fullerton, Calif.). Approximately 40,000 diverse small molecules are obtained from ChemDiv, Inc. (San Diego, Calif.), and 800 pharmacologically-active compounds are obtained from Prestwick Chemical Inc. (Washington, D.C.). Compound stocks are maintained in 100% DMSO (Sigma) at −20° C. For primary screening, each compound is at a final concentration of 6.25 μM and 1.0% DMSO. On day zero, 20 μl of complete medium containing the appropriate number of cells (150-300) is plated per well of a 384 well tissue culture plate (Fisher Scientific, Pittsburgh, Pa.) and incubated overnight at 37° C. with 5% CO₂ and 90% humidity. To minimize screening variability between cell lines, each 384 plate is divided into 4-96 well quadrants, with each quadrant harboring a specific cell line. Therefore a total of 80 compounds plus 16 DMSO (no-drug) controls are tested against 4 cell lines in each plate. Upon 20-24 hours post-seeding (day 1), compounds are prepared by serial dilution with complete medium to yield a concentration of 12.5 μM. Twenty μl of the 12.5 μM dilution are added to each well containing 20 μl of medium and cells, yielding the final screening concentration and a volume of 40 μl. Plates are incubated for an additional 3 days (day 2-5) until control cells (1.0% DMSO only) reach approximately 70-80% confluency upon visualization. On day six, 40 μl of lysis/detection solution containing 1.2% igepal CA-630 (Sigma, St. Louis, Mo.) and a 1:1000 dilution of SYBR Green I nucleic acid stain (Invitrogen, Rockville, Md.) is added to each well. Following an overnight incubation at 37° C., total fluorescence is measured using a fluorescence plate reader. The data is exported to a custom program (gift from Kenneth Kinzler) for determining growth inhibition. Compounds that show ≧50% growth inhibition compared to the DMSO-only controls' average are scored as “hits”. Compounds scored as hits are those that show cell-type specific growth inhibition and those that inhibit the growth of all cell lines at the screening concentration.

Secondary screens are performed as above except that each hit compound is tested using a concentration range extending from 18.75 μM down to 0.08 μM in 3 fold increments. Compounds found to specifically inhibit the growth of p21 −/− Tamoxifen resistant cells or MCF-7 spontaneously resistant cells when compared to parental control cell lines at 2 or more concentrations are repeated for confirmation. Using the same concentration range, confirmed secondary hits are tested for efficacy against multiple Tamoxifen resistant clones and cell lines.

Additional detail of the Examples are given in the Figures.

The entire contents of each of the following is hereby incorporated by reference:

Tamoxifen for early breast cancer: an overview of the randomised trials. Early Breast Cancer Trialists' Collaborative Group. Lancet, 1998 351: 1451-1467.
Osborne, C. K. Steroid hormone receptors in breast cancer management. Breast Cancer Res Treat, 1998 51: 227-238.
Fisher, B., Costantino, J. P., Wickerham, D. L., Redmond, C. K., Kavanah, M., Cronin, W. M., et al. Tamoxifen for prevention of breast cancer: report of the National Surgical Adjuvant Breast and Bowel Project P-1 Study. J Natl Cancer Inst, 1998 90: 1371-1388.
Osborne, C. K. Tamoxifen in the treatment of breast cancer. N Engl J Med, 1998 339: 1609-1618.
Wiebe, V. J., Osborne, C. K., Fuqua, S. A., and DeGregorio, M. W. Tamoxifen resistance in breast cancer. Crit Rev Oncol Hematol, 1993 14: 173-188.
Jordan, V. C. The secrets of selective estrogen receptor modulation: cell-specific coregulation. Cancer Cell, 2002 1: 215-217.
Bonneterre, J., Thurlimann, B., Robertson, J. F., Kirzakowski, M., Mauriac, L., Koralewski, P., et al. Anastrozole versus tamoxifen as first-line therapy for advanced breast cancer in 668 postmenopausal women: results of the Tamoxifen or Arimidex Randomized Group Efficacy and Tolerability study. J Clin Oncol, 2000 18: 3748-3757.
Mouridsen, H., Gershanovich, M., Sun, Y., Perez-Carrion, R., Boni, C., Monnier, A., et al. Superior efficacy of letrozole versus tamoxifen as first-line therapy for postmenopausal women with advanced breast cancer: results of a phase III study of the International Letrozole Breast Cancer Group. J Clin Oncol, 2001 19: 2596-2606.
Nabholtz, J. M., Buzdar, A., Pollak, M., Harwin, W., Burton, G., Mangalik, A., et al. Anastrozole is superior to tamoxifen as first-line therapy for advanced breast cancer in postmenopausal women: results of a North American multicenter randomized trial. Arimidex Study Group. J Clin Oncol, 2000 18: 3758-3767.
Goss, P. E., Ingle, J. N., Martino, S., Robert, N. J., Muss, H. B., Piccart, M. J., et al. A randomized trial of letrozole in postmenopausal women after five years of tamoxifen therapy for early-stage breast cancer. N Engl J Med, 2003 349: 1793-1802.
Coombes, R. C., Hall, E., Gibson, L. J., Paridaens, R., Jassem, J., Delozier, T., et al. A randomized trial of exemestane after two to three years of tamoxifen therapy in postmenopausal women with primary breast cancer. N Engl J Med, 2004 350: 1081-1092.
Bachman, K. E., Blair, B. G., Brenner, K., Bardelli, A., Arena, S., Zhou, S., et al. p21 (WAF1/CIP1) Mediates the Growth Response to TGF-beta in Human Epithelial Cells. Cancer Biol Ther, 2004 3: 221-225.
Cariou, S., Donovan, J. C., Flanagan, W. M., Milic, A., Bhattacharya, N., and Slingerland, J. M. Down-regulation of p21WAF1/CIP1 or p27Kip1 abrogates antiestrogen-mediated cell cycle arrest in human breast cancer cells. Proc Natl Acad Sci USA, 2000 97: 9042-9046.
Thomas, T. J., Faaland, C. A., Adhikarakunnathu, S., Watkins, L. F., and Thomas, T. Induction of p21 (CIP1/WAF1/SID1) by estradiol in a breast epithelial cell line transfected with the recombinant estrogen receptor gene: a possible mechanism for a negative regulatory role of estradiol. Breast Cancer Res Treat, 1998 47: 181-193.
Kobayashi, M., Yamauchi, Y., Tanaka, A., and Shimamura, S. Improved dicistronic mRNA expression vectors for efficient selection of transfectants highly expressing foreign genes. Biotechniques, 1996 21: 398-402.
Jones, S. E. Fulvestrant: an estrogen receptor antagonist that downregulates the estrogen receptor. Semin Oncol, 2003 30: 14-20.
Gohring, U. J., Bersch, A., Becker, M., Neuhaus, W., and Schondorf, T. p21(waf) correlates with DNA replication but not with prognosis in invasive breast cancer. J Clin Pathol, 2001 54: 866-870.
Han, F., Miksicek, R., Clarke, R., and Conrad, S. E. Expression of an estrogen receptor variant lacking exon 3 in derivatives of MCF-7 cells with acquired estrogen independence or tamoxifen resistance. J Mol Endocrinol, 2004 32: 935-945.
Torrance, C. J., Agrawal, V., Vagelstein, B., and Kinzler, K. W. Use of isogenic human cancer cells for high-throughput screening and drug discovery. Nat Biotechnol, 2001 19: 940-945.

What is claimed is:

1. A composition, comprising:
at least one compound selected from the group consisting of a compound having formula (1), a salt thereof, a prodrug thereof, a compound having formula (II), a salt thereof, a prodrug thereof, and a combination thereof; and
at least one pharmaceutically acceptable carrier;
wherein the composition is suitable for administration to a mammal;
wherein the compound having formula (I) is: 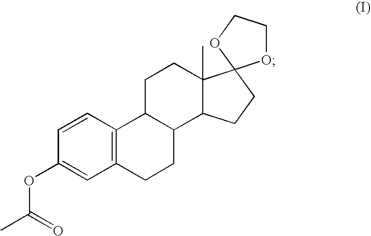
and wherein the compound having formula (II) is:

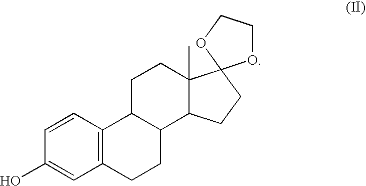

2. The composition of claim 1, wherein the compound is the compound having formula (I), a salt thereof, a prodrug thereof, or a combination thereof.

3. The composition of claim 1, wherein the compound is the compound having formula (II), a salt thereof, a prodrug thereof, or a combination thereof.

4. The composition of claim 1, wherein the compound having formula (I) has the following structure:

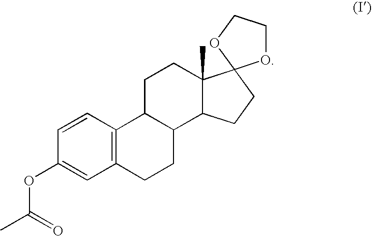

5. The composition of claim 1, wherein the compound having formula (II) has the following structure:

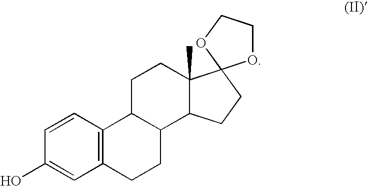

6. The composition of claim 1, wherein the pharmaceutically acceptable carrier comprises at least one selected from the group consisting of water, physiological saline, ethanol, aqueous ethanol, dimethyl sulfoxide, castor oil, benzyl alcohol, benzyl benzoate, albumin, polyethylene glycol, cellulose, fatty acid, methylcellulose, dextrose, glycerol, mannitol, lactose, starch, magnesium stearate, sodium saccharin, magnesium carbonate, and a combination thereof.

7. A method, comprising:
administering, to a mammal, at least one compound selected from the group consisting of a compound having formula (I), a salt thereof, a prodrug thereof, a compound having formula (II), a salt thereof, a prodrug thereof, and a combination thereof;
wherein the compound having formula (I) is: 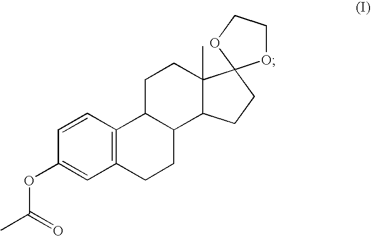
and wherein the compound having formula (II) is:

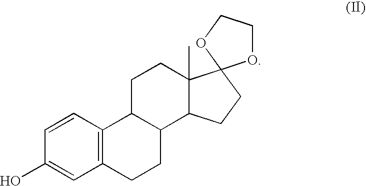

8. The method of claim 7, wherein the mammal is known to have a hormonal dependent malady.

9. The method of claim 7, wherein the mammal is known to have a hormonal dependent malady selected from the group consisting of cancer, Tamoxifen-resistant cancer, Tamoxifen resistant breast cancer, hormone-dependent breast cancer, hormone-dependent endometrial cancer, hormone-dependent ovarian cancer, estrogen receptor expressing cancer, progesterone receptor expressing cancer, vaginitis, vaginal dryness, post-menopausal vaginitis, osteoporosis, cardiovascular disease, decreased fertility, infertility, and a combination thereof.

10. The method of claim 7, wherein the mammal is in need of a contraceptive.

11. The method of claim 7, wherein the mammal is a human.

12. The method of claim 7, wherein the mammal is a female human.

13. The method of claim 7, wherein the compound is administered in the form of a composition, said composition comprising:
the at least one compound selected from the group consisting of a compound having formula (I), a salt thereof, a prodrug thereof, a compound having formula (II), a salt thereof, a prodrug thereof, and a combination thereof; and
at least one pharmaceutically acceptable carrier.

14. The method of claim 7, wherein the compound is the compound having formula (I), a salt thereof, a prodrug thereof, or a combination thereof.

15. The method of claim 7, wherein the compound is the compound having formula (II), a salt thereof, a prodrug thereof, or a combination thereof.

16. The method of claim 7, wherein the compound having formula (I) has the following structure:

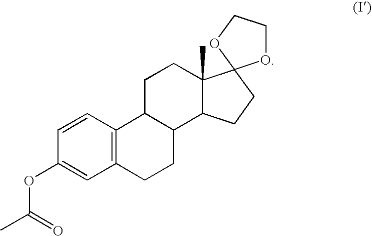

17. The method of claim 7, wherein the compound having formula (II) has the following structure:

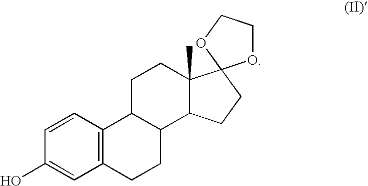

18. The method of claim 13, wherein the pharmaceutically acceptable carrier comprises at least one selected from the group consisting of water, physiological saline, ethanol, aqueous ethanol, dimethyl sulfoxide, castor oil, benzyl alcohol, benzyl benzoate, albumin, polyethylene glycol, cellulose, fatty acid, methylcellulose, dextrose, glycerol, mannitol, lactose, starch, magnesium stearate, sodium saccharin, magnesium carbonate, and a combination thereof.

19. A method for manufacturing a medicament suitable for treating a mammal having a hormonal dependent malady, comprising contacting the compound of claim 1 with at least one pharmaceutically acceptable carrier to form a composition suitable for administration to the mammal.
